(19) United States
(12) Patent Application Publication
(10) Pub. No.: US 2024/0238256 A1
(43) Pub. Date: Jul. 18, 2024

(54) NITAZOXANIDE IN THE TREATMENT OF SEPSIS

(71) Applicant: GENFIT, Loos (FR)

(72) Inventors: Vanessa LEGRY, Emmerin (FR); Rémy HANF, Lille (FR); Simon DEBAECKER, Pont-a-Marcq (FR)

(73) Assignee: GENFIT, Loos (FR)

(21) Appl. No.: 18/289,511

(22) PCT Filed: May 10, 2022
(86) PCT No.: PCT/EP2022/062714

§ 371 Date: Nov. 3, 2023

(30) Foreign Application Priority Data
May 11, 2021 (EP) 21305616.1

(51) Int. Cl. A61K 31/426; A61K 31/407; A61P 31/04

(52) U.S. Cl. CPC A61K 31/426; A61K 31/407; A61P 31/04

(57) ABSTRACT

The present invention relates to a compound selected from nitazoxanide, tizoxanide and tizoxanide glucuronide, for use in a method for the treatment of sepsis in a subject in need thereof.

The present invention relates to nitazoxanide, tizoxanide or tizoxanide glucuronide for use in the treatment or prevention of sepsis.

### BACKGROUND OF THE INVENTION

Sepsis is a dysregulated immune response to an infection that leads to organ dysfunction. It develops as the result of a complex, dysregulated host response to infection, a bacterial infection in most cases. This dysregulated host response is characterized not only by increased inflammation but also by immune suppression. The effects of this inappropriate response to infection lead to cellular dysfunction and, ultimately, organ failure. Single organ dysfunction in sepsis is rare, and several organs are usually affected. Mortality in patients with sepsis correlates with the number of organs that are affected.

Many patients with sepsis develop circulatory failure that results in abnormal cellular oxygen metabolism. Abnormal cellular oxygen metabolism manifests as an increase in blood lactate levels, typically to values >2 mEq per litre. Patients who require vasopressors to maintain a minimum mean arterial pressure despite adequate volume resuscitation and who have raised blood lactate levels are clinically diagnosed as having septic shock.

Sepsis, the systemic inflammatory response to infection, is manifested by two or more of the criteria that define systemic inflammatory response syndrome. Severe sepsis is a condition complicated by organ dysfunction and septic shock (hypotension despite adequate fluid resuscitation). The end of the spectrum is multiple organ dysfunction syndrome, which is defined as the presence of altered organ function in an acutely ill patient and homeostasis that cannot be maintained without intervention.

Sepsis and the resultant multiple organ failure that it induces are the most common causes of death in many intensive care units. It is estimated that 750,000 cases of severe sepsis occur each year in the United States, with a high mortality rate. Sepsis is now the 12th most common cause of death in America. As a matter of fact, sepsis is defined as “the systemic inflammatory response syndrome due to infection” and reflects the concept that sepsis is the result of an uncontrolled inflammatory cascade.

Increasing evidence now suggests that extensive apoptotic death results in immune cell depletion and may compromise the ability of the patient to eradicate infections.

Apoptosis represents the execution of an ATP-dependent death program that is often initiated by death receptor ligation, leading to a caspase activation cascade, including activation of caspase-9 and subsequent activation of effector caspases. Once active, caspase-9 can directly cleave and activate caspase-3 and caspase-7.

The caspase gene family consists of 15 mammalian members classified based on the structure and function of their prodomains. The caspase family can be divided into two functional subgroups based on their roles. Inflammatory caspases (caspase-1, -4, -5, 11, -12, -13 and -14) play a role in cytokine maturation and inflammatory responses. The caspases involved in apoptosis are further divided into two functional subgroups, initiator of apoptosis caspases (caspase-2, -8, -9, -10 and -15) and effector caspases (caspase-3, 6, 7).

Effector caspases are responsible for initiating the hallmarks of the degradation phase of apoptosis, including DNA fragmentation, cell shrinkage and membrane blebbing.

Moreover, there is an association between serum caspase 3 levels at moment of severe sepsis diagnosis and mortality in septic patients. Serum caspase 3 levels could then be used as prognostic biomarker. Increased caspase 3 activity has been found in different body sites in animal models of sepsis. In addition, higher caspase 3 activity has been found in lymphocytes of septic patients than of healthy controls and in spleen of septic patients than in nonseptic patients.

Current treatment for sepsis aims to limit the development of organ dysfunction by providing rapid control of infection, haemodynamic stabilization and organ support when possible to ensure recovery of organ function. But treatment of sepsis and septic shock remains a substantial unmet medical need.

NTZ (nitazoxanide, [2-[(5-nitro-1,3-thiazol-2-yl)carbamoyl]phenyl]ethanoate), first described in 1975, was shown to be highly effective against anaerobic protozoa, helminths, and a wide spectrum of microbes including both anaerobic and aerobic bacteria. NTZ is a medicament authorized in the United States for the treatment of diarrhea caused by the protozoan parasites Cryptosporidium parvum and Giardia intestinalis.

NTZ can also confer antiviral activity and was also shown to have broad anticancer properties by interfering with crucial metabolic and prodeath signaling pathways.

It is herein surprisingly shown that NTZ can be used for the treatment of sepsis in a subject in need thereof.

### SUMMARY OF THE INVENTION

The invention stems from the surprising observation that NTZ improves survival in a preclinical model of sepsis. The inventors have also shown that tizoxanide (TZ), the active metabolite of NTZ, directly protects hepatocytes from cytokine-induced cell death by inhibiting caspase activity.

Accordingly, the invention relates to NTZ, TZ, TZ glucuronide (TZG), or a pharmaceutically acceptable salt thereof, for use in a method of treatment of sepsis.

The invention more particularly relates to a compound selected from NTZ, TZ and TZG, for use in a method for the treatment of sepsis in a subject in need thereof. In a particular embodiment, said compound is NTZ.

In a particular embodiment, said sepsis is caused by a bacterial infection.

In another particular embodiment, the compound is used to protect vital organs by inhibiting cytokine-induced apoptosis that occurs during sepsis. In yet another embodiment, the compound is used to protect against cytokine-induced cell death by inhibiting caspase activity.

In a particular embodiment, said subject suffers from or is at risk of sepsis with multiple organ failure. In another embodiment, said subject suffers from or is at risk of septic shock.

In another embodiment, the compound is for use to slow or stop the progression of sepsis.

In yet another embodiment, said compound is for use as a single active agent in said method.

Alternatively, in another embodiment, said compound is for use in combination with an antimicrobial agent, such as an antibiotic, in said method. In a particular embodiment, said antimicrobial agent is a carbapenem antibiotic, such as ertapenem.

### BRIEF DESCRIPTION OF THE DRAWINGS

FIG. 1: NTZ treatment improves survival after CLP surgery

Survival curves after CLP surgery in mice treated or not (vehicle) with NTZ

P=0.07 for the comparison of the survival curves between NTZ and vehicle groups using the Log rank Mantel-Cox test

FIG. 2: TZ inhibits caspase 3/7 activity induced by TNFα in HepG2

*, ** , *** for p<0.05, p<0.01 and p<0.001, respectively, for the comparison between TNFα or staurosporine versus untreated (A), and between TZ versus vehicle (B) using ANOVA and Fisher's LSD test for multiple comparison

### for p<0.001 using Student T test

FIG. 3: TZ pretreatment inhibits caspase 3/7 activity induced by staurosporine in HepG2 cells

A. Effect of staurosporine on caspase 3/7 activity in HepG2 cells (n=24). Student t-test was used to assess statistical significance. ***p<0.001

B. Effect of TZ pretreatment on staurosporine-induced apoptosis in HepG2 cells (n=8 to 24). Cells were pretreated for 16 h with TZ before addition of staurosporine. One-way ANOVA with Dunnett test for multiple testing was used to assess statistical significance (TZ-treated cells compared to untreated cells). ***p<0.001

FIG. 4: TZ and NTZ treatments concomitant to staurosporine inhibit caspase 3/7 activity in HepG2 cells

A. Effect of TZ on staurosporine-induced apoptosis in HepG2 cells (n=6). TZ and staurosporine were added simultaneously before Caspase 3/7 activity measurement

B. Effect of NTZ on staurosporine-induced apoptosis in HepG2 cells (n=6). NTZ and staurosporine were added simultaneously before Caspase 3/7 activity measurement.

For A and B, one-way ANOVA with Dunnett test for multiple testing was used to assess statistical significance (TZ or NTZ-treated cells compared to untreated cells). **p<0.01, *** p<0.001

FIG. 5: NTZ with or without pretreatment improves survival after CLP-induced sepsis

A. Study synopsis—the white squares indicate no treatment (control mice) and the black triangles indicate the BID treatment of mice with NTZ

B. Survival curves of control mice, mice that received NTZ with a 3-day pretreatment, and mice that received NTZ from the day of CLP surgery. Survival curves were compared between groups using the Gehan-Breslow-Wilcoxon. **p<0.01, ***p<0.001

FIG. 6: NTZ with or without pretreatment improves survival rates 7 days after CLP-induced sepsis

Survival rates at the end of the study in mice treated with vehicle, NTZ 3-day pretreatment, or NTZ treatment

FIG. 7: NTZ improves welfare scores in mice with CLP-induced sepsis

Evolution of the 6 individual parameters considered to evaluate animal welfare during the 4 days following CLP surgery in mice treated with vehicle, NTZ 3-day pretreatment, or NTZ treatment. The severity was evaluated from 0 (no sign) to 3 (more severe).

FIG. 8: NTZ administration after the induction of sepsis is efficient to improve survival

A. Study synopsis. The white squares indicate no treatment (control mice) and the triangles indicate the treatment of mice with NTZ (1 triangle=once daily or QD, two triangles=twice daily or BID)

B. Survival curves of control mice, mice that received NTZ BID-1 h before and 3.5 h after CLP, and mice that received NTZ only 3.5 h after CLP surgery. Survival curves were compared between groups using the Gehan-Breslow-Wilcoxon. **p<0.01, ***p<0.001

C. Survival rates at the end of the study in control mice, mice that received NTZ BID-1 h before and 3.5 h after CLP, and mice that received NTZ only 3.5 h after CLP surgery

FIG. 9: NTZ administration after the induction of sepsis is efficient to improve welfare scores

Evolution of the 6 individual parameters considered to evaluate animal welfare during the 4 days following sepsis induction in control mice, mice that received NTZ BID-1 h before and 3.5 h after CLP, and mice that received NTZ only 3.5 h after CLP surgery. The severity was evaluated from 0 (no sign) to 3 (most severe).

### DETAILED DESCRIPTION OF THE INVENTION

The present invention relates to NTZ or TZ(G) for use in the treatment or prevention of sepsis.

The term “subject” or “patient” as used herein refers to a mammal, preferably a human.

As mentioned above, the term “sepsis” as used herein refers to a deleterious systemic inflammatory response to infection, formally defined as the presence of infection together with systemic manifestations of infection. The term sepsis as used herein encompasses sepsis, at any degree of severity, and complications thereof such, such as sepsis with multiple organ failure and septic shock.

In a particular embodiment of the invention, the subject suffers or is at risk of suffering from sepsis or complications thereof.

In another particular embodiment, the subject suffers from sepsis caused by one or more microbial species. In particular, the subject may suffer from sepsis caused by a bacterial, fungal or viral infection. In yet another embodiment, said sepsis is caused by a bacterial infection.

In a particular embodiment, the method of treatment or prevention consists of the administration of NTZ, TZ or TZG as a single active ingredient.

The term “treatment”, as used herein, relates to both therapeutic measures and prophylactic or preventative measures, wherein the goal is to prevent or slow down (lessen) an undesired physiological change or disorder. Beneficial or desired clinical results include, but are not limited to, alleviation of symptoms, stabilizing pathological state (specifically not worsening), slowing down or stopping the progression of the disease, improving or mitigating the pathological. Particularly, for the purpose of the present invention, treatment is directed to slow the progression of sepsis and reduce the risk of further complications. It can also involve prolonging survival in comparison with the expected survival if the treatment is not received. In a particular embodiment, NTZ, TZ(G) or a pharmaceutically acceptable salt thereof is used to reduce the mortality associated to sepsis. NTZ, TZ(G) or a pharmaceutically acceptable salt thereof can also be used to slow or stop the progression of sepsis. In particular, NTZ, TZ(G) or a pharmaceutically acceptable salt thereof is used to prevent the progression of sepsis, in particular to prevent the progression of sepsis to septic shock in a subject suffering from sepsis. In another embodiment, NTZ, TZ(G) or a pharmaceutically acceptable salt thereof is used to prevent organ failure, in particular multiple organ failure, in a subject suffering from sepsis.

In the context of the present invention NTZ, TZ(G), or a pharmaceutically acceptable salt thereof is administered to the subject, in a therapeutically effective amount. In a particular embodiment, NTZ or TZ, or a pharmaceutically acceptable salt thereof is administered. In a further embodiment, the subject is administered with NTZ or a pharmaceutically acceptable salt thereof, in particular with NTZ.

A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

NTZ, TZ(G) or a pharmaceutically acceptable salt thereof can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art.

These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

NTZ or TZ(G) can be in the form of pharmaceutically acceptable salts particularly acid or base salts compatible with pharmaceutical use. Salts of NTZ and TZ(G) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

NTZ, TZ(G), or a pharmaceutically acceptable salt thereof may be administered by different routes and in different forms. For example, the compound(s) may be administered via a systemic way, per os, parenterally, by inhalation, by nasal spray, by nasal instillation, or by injection, such as for example intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally.

The frequency and/or dose relative to the administration can be adapted by one of ordinary skill in the art, in function of the patient, the pathology, the form of administration, etc. Typically, NTZ or TZ(G) can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. In a particular embodiment, the NTZ, TZ(G), or a pharmaceutically acceptable salt thereof, is administered at a dose of about 1000 mg/day, in particular at 1000 mg/day. In a particular embodiment, NTZ, TZ(G), or a pharmaceutically acceptable salt thereof, is administered orally at a dose of about 1000 mg/day, in particular at 1000 mg/day, in particular as a tablet. Administration can be performed daily or even several times per day, if necessary. In one embodiment, the compound is administered at least once a day, such as once a day, twice a day, or three times a day. In a particular embodiment, the compound is administered once or twice a day. In particular, oral administration may be performed once a day, during a meal, for example during breakfast, lunch or dinner, by taking a tablet comprising the compound at a dose of about 1000 mg, in particular at a dose of 1000 mg. In another embodiment, a tablet is orally administered twice a day, such as by administering a first tablet comprising the compound at a dose of about 400 mg, about 500 mg or about 600 mg, in particular at a dose of 500 mg, during one meal, and administering a second tablet comprising the compound at a dose of about 500 mg, in particular at a dose of 500 mg, during another meal the same day.

In another particular embodiment, the administration of NTZ or TZ(G) is performed in combination with another active ingredient, preferably with an antimicrobial agent such as an antibiotic, an antifungal or an antiviral. Of course, the most suitable antimicrobial agent will be selected depending on the organism or virus responsible for the infection, as is well known in the art. In a particular embodiment, the sepsis is caused by a bacterial infection, and the antimicrobial is an antibiotic. Antibiotics useful in the treatment of bacterial infections are well known in the art. Illustrative antibiotic families include, without limitation, beta-lactam antibiotics (such as penicillins), tetracyclines, cephalosporins, quinolones, lincomycins, macrolides, sulfonamides, glycopeptides, aminoglycosides and carbapenems. In a particular embodiment, NTZ or TZ(G) can be combined to an antibiotic of the carbapenem family, such as ertapenem.

NTZ, or TZ(G), and the antimicrobial agent can be administered to the subject in the same or separate pharmaceutical compositions. In a particular embodiment, the invention provides a pharmaceutical composition comprising NTZ or TZ(G), an antimicrobial agent and a pharmaceutically acceptable excipient. This pharmaceutical composition can be used in the method of the invention, for the treatment or prevention of sepsis. In another embodiment, the invention provides a method wherein
a first pharmaceutical composition comprising NTZ or TZ(G) and a pharmaceutically acceptable excipient; and a second pharmaceutical composition comprising the antimicrobial agent;
are both administered to the subject for the treatment or prevention of sepsis.

The first and second pharmaceutical compositions can be used simultaneously, separately or sequentially (i.e. the first pharmaceutical composition can be administered before or after the second pharmaceutical composition). As such, the invention also provides a kit-of-parts comprising:
a first pharmaceutical composition comprising NTZ or TZ(G) and a pharmaceutically acceptable excipient; and
a second pharmaceutical composition comprising the antimicrobial agent;
for simultaneous, separate or sequential use in the treatment or prevention of sepsis.

The following examples serve to illustrate the invention and must not be considered as limiting the scope thereof.

### EXAMPLES

### Example 1: NTZ Improves Survival in a Preclinical Model of Sepsis

Polymicrobial sepsis induced by cecal ligation and puncture (CLP) is characterized by dysregulated systemic inflammatory responses followed by immunosuppression. The CLP model in mice mimics the progression and features of human sepsis and is thus also useful to determine whether a drug would be efficient in the treatment of prevention of transition from sepsis to septic shock.

This study aims to investigate the efficacy of NTZ in CLP model in C57BL6J (BL6) male mice. The efficacy of the test compound was evaluated based on the survival rate of the animals within the study period.

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

### Cecal Ligation and Puncture Surgery

C57BL6J male mice (supplier Janvier—France) at 9 weeks of age and weighing 23-25 g on arrival were anesthetized with 250 UL of xylazine/ketamine solution (6.75 mg/kg for ketamine (Imalgene, Boehringer, Germany) et 2.5 mg/kg for xylazine (Rompund 2%, Bayer, Germany)) by intraperitoneal route. A 1-1.5 cm abdominal midline incision was made, and the cecum was located and tightly ligated at half the distance between distal pole and the base of the cecum with 4-0 silk suture (mild grade). The caecum was punctured through-and-through once with a 21-gauge needle from mesenteric toward antimesenteric direction after medium ligation. A small amount of stool was extruded to ensure that the wounds were patent. Then the cecum was replaced in its original position within the abdomen, which was closed with sutures and wound clips. Mice were followed for body weight evolution and mortality rate until Day 7.

### Treatment Scheme

NTZ (Interchim, France) was administered by oral gavage at 50 mg/kg BID. NTZ treatment was initiated 3 days before CLP. The day of the surgery, NTZ was given once (50 mg/kg) 1 h before CLP and then a second time when animals have recovered from anesthesia (50 mg/kg). BID treatments was then pursued daily until the end of the study (n=15). Mice receiving NTZ vehicle (carboxymethylcellulose (#C4888, Sigma-Aldrich, Germany) BID served as controls (n=10).

Ertapenem 10 mg/kg (ORB134782/PO8952, Interchim/Biorbyt) was used as pharmacological reference control and was administered by intraperitoneal route, 1 h before surgery at Day 0 and pursued daily after CLP surgery (n=10).

### Results

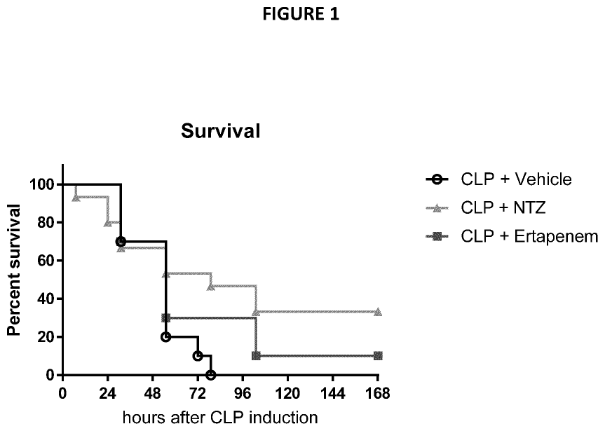

CLP induced 100% mortality 3 days after surgery in the group of mice receiving the vehicle only (FIG. 1). On the contrary, 47% of mice treated with NTZ were still alive 3 days after surgery, and 33% of mice even survived 7 days after intervention. Noteworthy, NTZ even better improved survival than the pharmacological reference Ertapenem, which only saved 10% of mice at the end of the study.

In conclusion, NTZ has a beneficial effect on survival rate in CLP induced polymicrobial sepsis in mice.

### Example 2: NTZ Protects Hepatocytes from Cytokine-Induced Apoptosis

The uncontrolled cytokine storm developing during the transition from sepsis to septic shock induces cell death in the different tissues which can jeopardize the functioning of vital organs such as the liver.

This study aims to investigate the efficacy of NTZ to protect hepatocytes from cellular damages, in particular apoptosis induced by cytokines.

### Evaluation of TNFα-Induced Apoptosis in Human Hepatocytes

In order to evaluate the effect of NTZ on human hepatocytes that undergo a cellular stress induced by cytokines, the human hepatoblastoma-derived HepG2 cell line (#85011430, ECACC, UK) was cultured with or without TZ, the active metabolite of NTZ, in high-glucose

DMEM medium (#41965, Gibco, France) supplemented with 10% of fetal bovine serum (FBS, #10270, Gibco), 1% penicillin/streptomycin (#15140, Gibco), 1% sodium pyruvate (#11360, Gibco) and 1% MEM non-essential amino acids (#11140, Gibco) in a 5% CO2 incubator at 37° ° C.

To evaluate caspase 3/7 activity, which is a surrogate marker of apoptosis, 5×10⁴ cells were plated in a 96-well plate (Thermo Fischer, Germany). After cell adherence (8 hours), cells were pre-treated with a dose range of 0.3 to 3 μM of TZ (Interchim, France), in FBS-deprived cell culture medium for 16 h. Thereafter, tumor necrosis factor α (TNFα) (#C6378, Promocell, Germany) was added to the wells at the dose of 10 or 30 ng/ml for additional 24 hours. Staurosporine (10 μM) (#19-123MG, Sigma-Aldrich, Germany) was used as a reference to induce apoptosis. Cells were incubated with staurosporine 3 hours before Caspase activity measurement.

Caspase 3/7 activity was measured using Caspase Glow™ 3/7 assay (#G8093, Promega, USA). Luminescence was measured using a Spark microplate reader (#30086376, Tecan, USA). The amount of luminescence (RLU) directly correlates with caspase 3/7 activity.

### Results

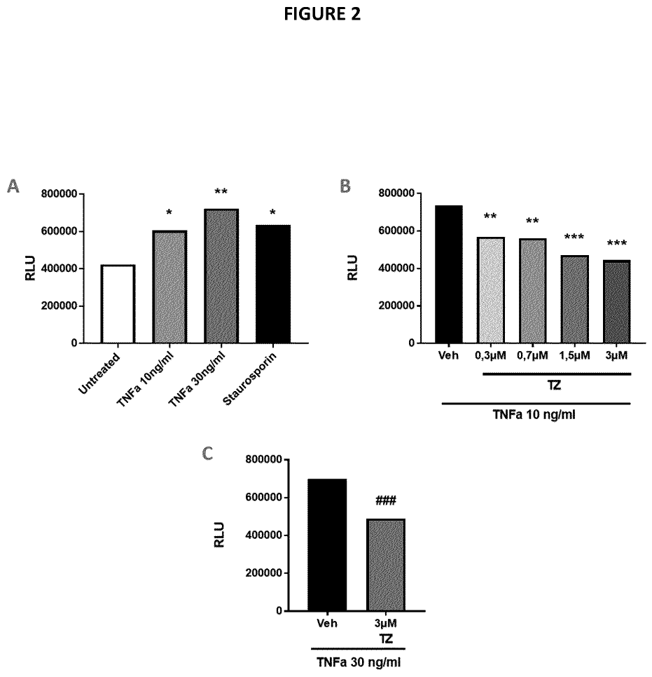

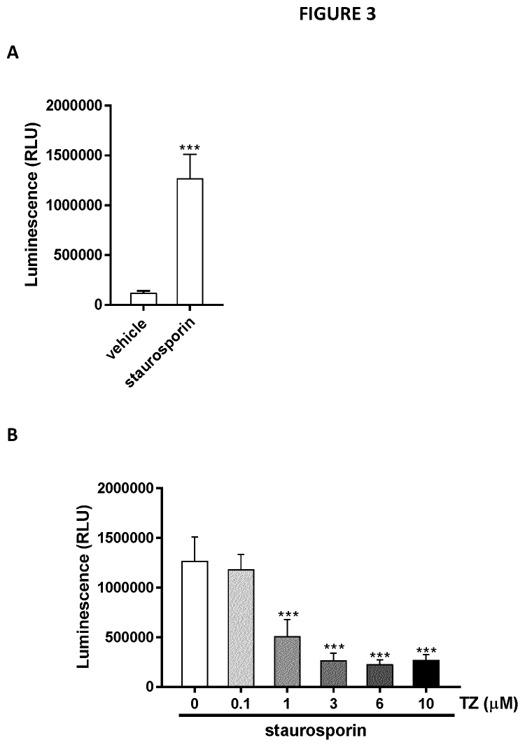

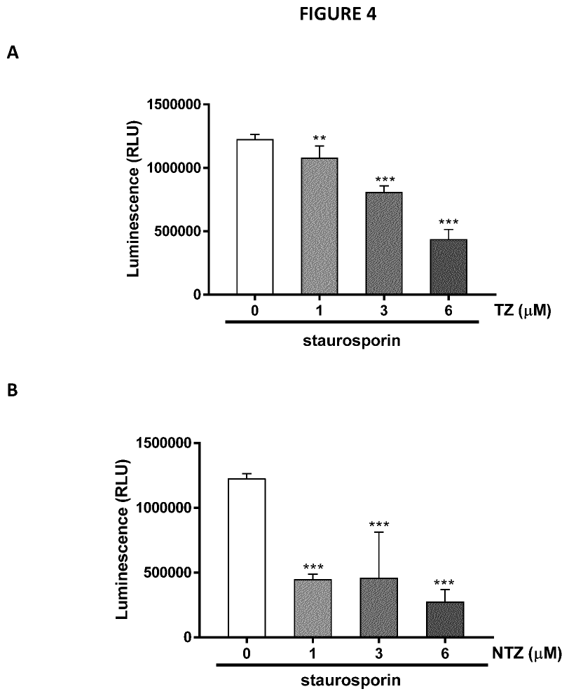

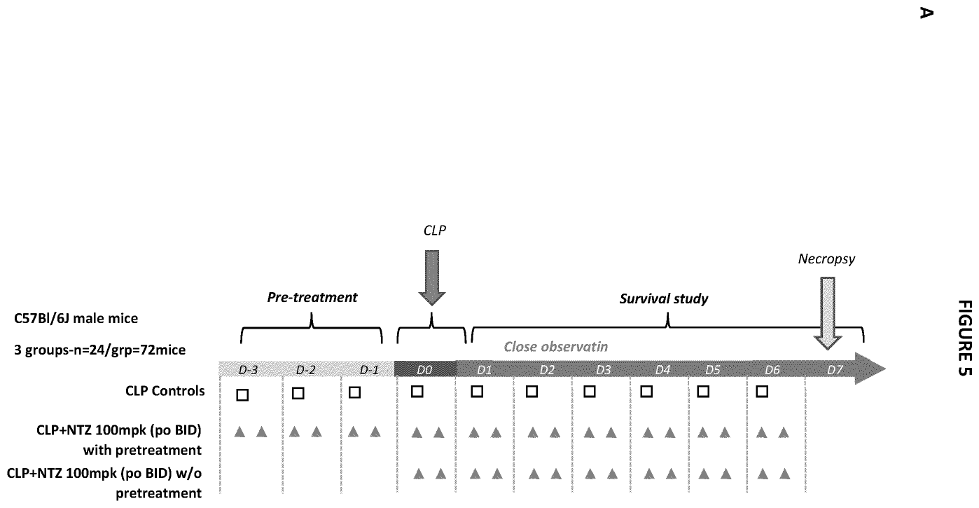

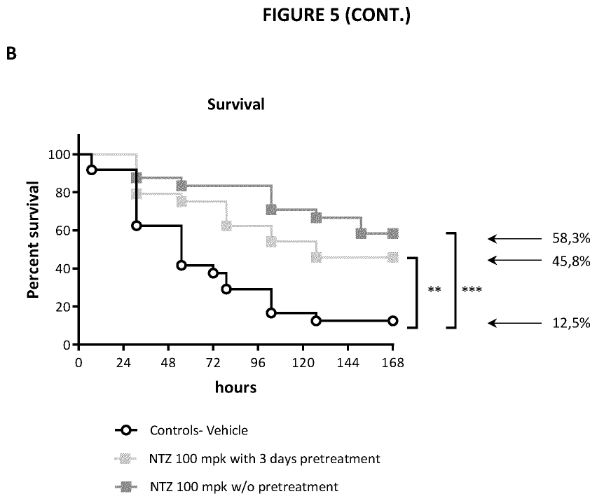

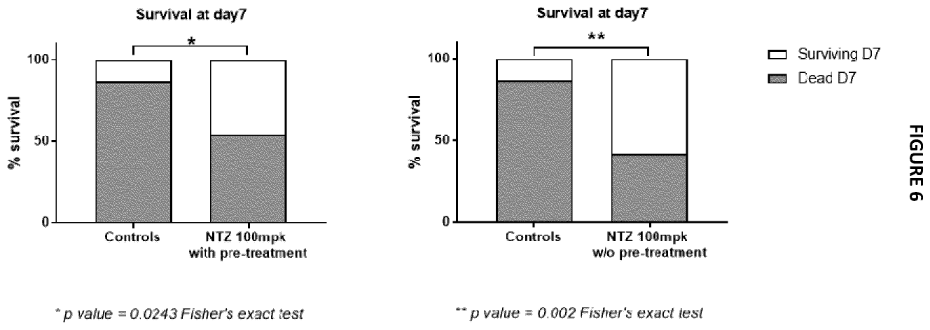

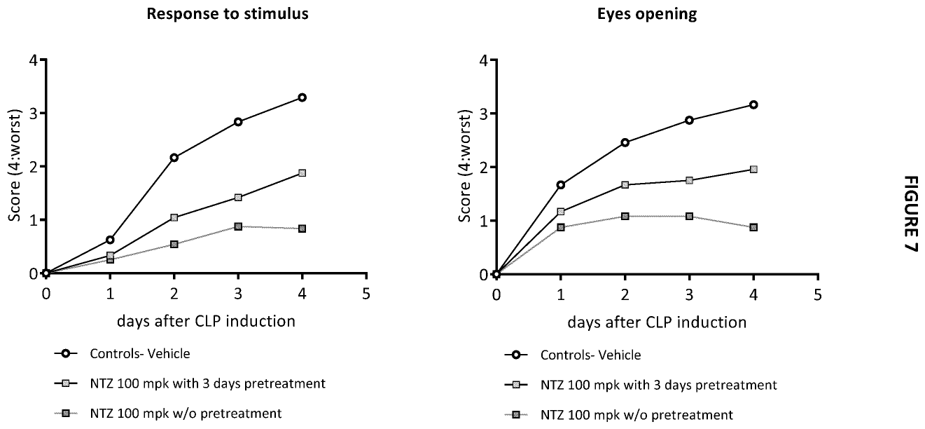

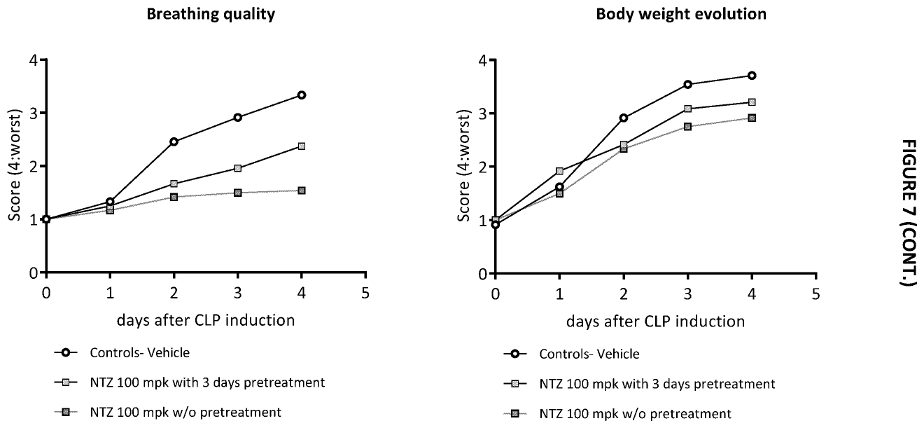

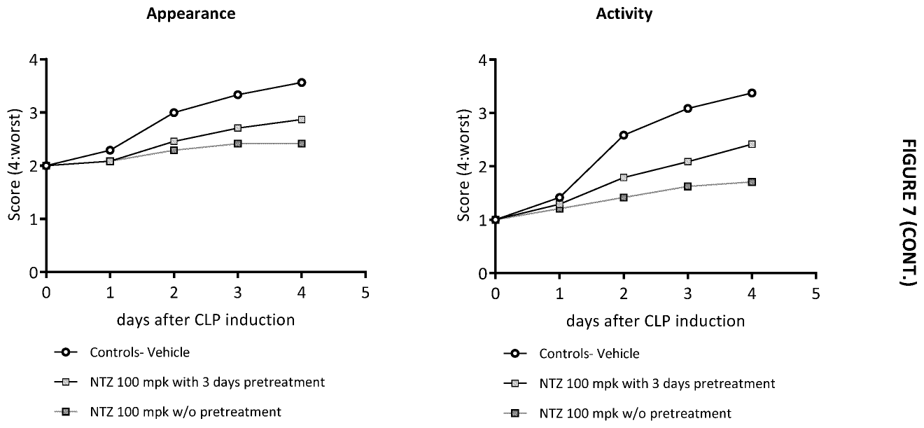

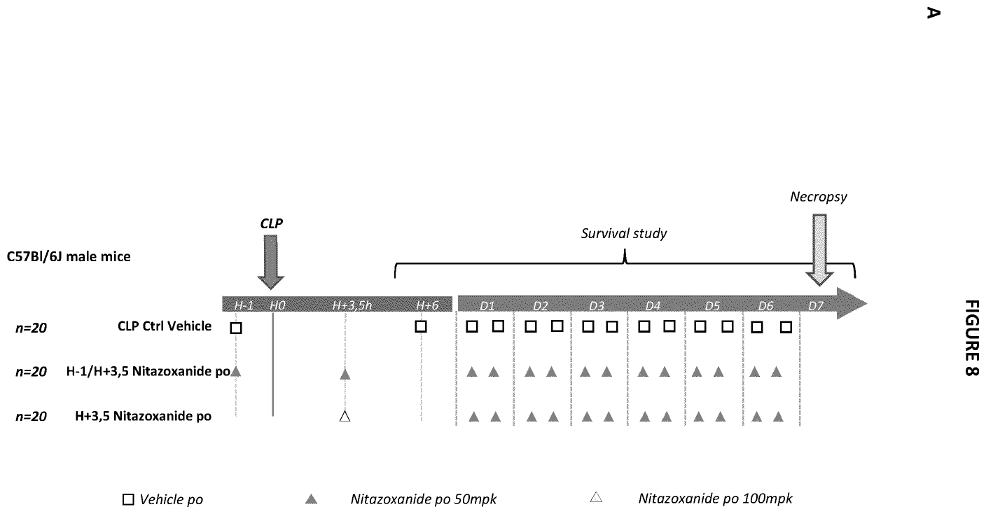

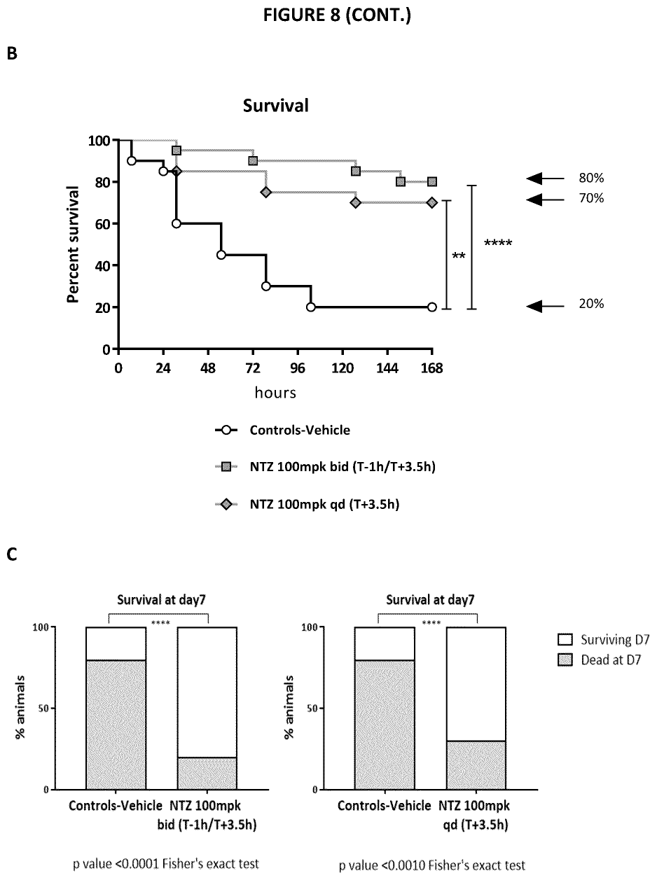

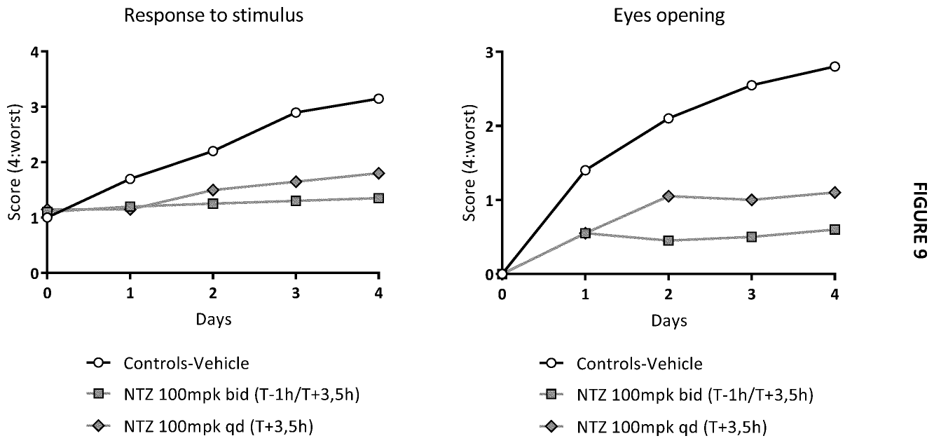

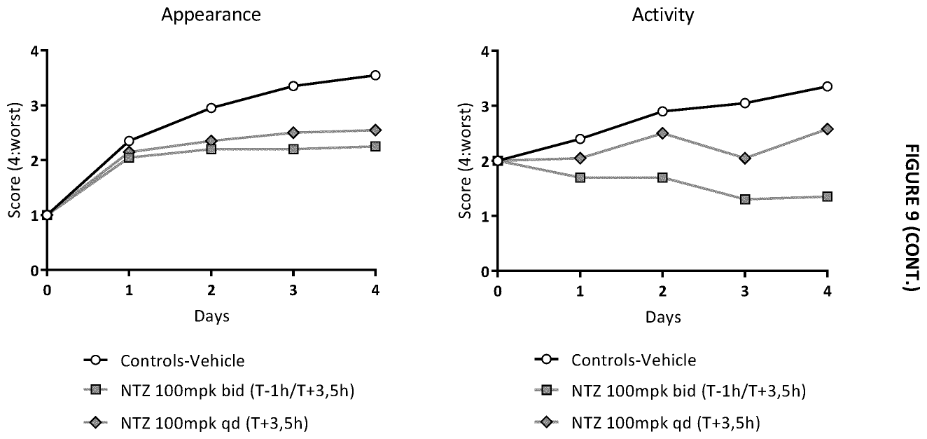

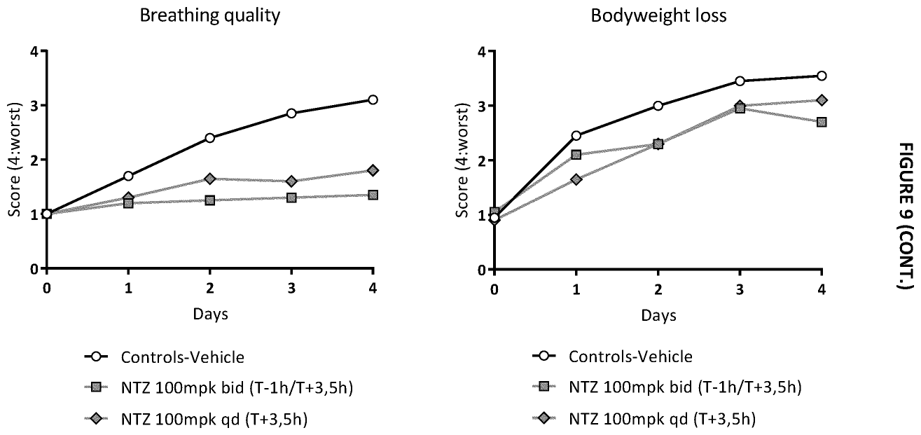

Incubation of HepG2 with TNFα induced apoptosis, as shown by increased caspase 3/7 activity by 1.5-fold with 10 ng/ml TNFα and 1.7-fold with 30 ng/ml, an effect size comparable to the apoptosis inducer staurosporine (FIG. 2A). Treatment with TZ remarkably reduced caspase activity in a dose response manner in presence of 10 ng/ml TNFα (FIG. 2B), reaching 40% inhibition at the dose of 3 μM TZ. Noteworthy this effect was confirmed with the higher TNFα dose (FIG. 2C). These results show that TZ directly protects hepatocyte from cell death by inhibiting caspase activity.

### Example 3: Direct and Rapid Effects of NTZ on Apoptosis in Hepatocytes

This study aims to investigate the efficacy of NTZ and its active metabolite, TZ, with or without pretreatment, to protect hepatocytes from cellular damages induced by a strong inducer of apoptosis, ie staurosporine (a protein kinase inhibitor that activates caspases).

### Protocol

The human hepatoblastoma-derived HepG2 cell line was cultured as described in Example 2. Caspase 3/7 activity was assessed in 1.5×10⁴ cells plated in a 384-well plate (#781080, Greiner, France). After cell adherence (8 hours), cells were serum starved for 16 h with or without TZ, the active metabolite of NTZ. Thereafter, cells were treated with a high dose of staurosporine (30 μM, #569397, Sigma-Aldrich, Germany) supplemented with 0.1 to 10 μM of TZ (#RP253, Interchim) or 1 to 6 μM NTZ (#RQ550, Interchim, France) for 4 hours before cell lysis and caspase activity measurement. Caspase 3/7 activity was measured as previously described.

### Results

Incubation of HepG2 cells with staurosporine strongly induced apoptosis, as shown by an increase of caspase 3/7 activity by 11-fold (FIG. 3A). When used as a pretreatment, TZ remarkably reduced caspase activity induced by staurosporine in a dose dependent manner, reaching 82% inhibition at a dose of 6 μM TZ (FIG. 3B). Interestingly, the concomitant addition of staurosporine with 6 μM TZ without TZ pretreatment also decreased caspase activity by 64% (FIG. 4A). In this condition, NTZ showed a similar effect with 78% inhibition of caspase activity (FIG. 4B). These results show that both NTZ and its active metabolite TZ are potent inhibitors of apoptosis, protecting hepatocytes from cell damages that notably occur during sepsis.

### Example 4: NTZ Improves Survival of CLP-Mice without NTZ Pretreatment

Given the rapid effects of NTZ observed in vitro, we investigated the efficacy of NTZ to protect from sepsis in the CLP model in 2 curative settings.

Polymicrobial sepsis was induced by CLP surgery in C57BL6J mice, as described in Example 1. NTZ was prepared as previously described and administrated per os at 100 mg/kg/day BID either starting 3 days before CLP (3 day pretreatment) or starting on the same day as the CLP surgery (without pretreatment) as shown in FIG. 5A. C57BI/6J male mice (8 week-old, Janvier, France) were divided into 3 groups of 24 mice each, after 7 days acclimation:
Group 1 received vehicle for 3 days before surgery and for 6 days after CLP surgery.
Group 2 received nitazoxanide (NTZ) for 3 days before surgery and then for 6 days after CLP surgery (pretreatment).
Group 3 received vehicle for 3 days before surgery and then NTZ for 6 days after CLP surgery (without pretreatment).

Treatments with NTZ were made twice daily at 9 am and 5 μm. The day of the CLP surgery (Day 0), mice received NTZ or vehicle one hour before being anaesthetized (groups 2 and 3).

Welfare scoring of severity of murine sepsis model has been published to harmonize human end points and also to normalize grades observed between animals throughout experiments (Shrum B, Anantha R V, Xu S X et al. A robust scoring system to evaluate sepsis severity in an animal model. BMC Res Notes 2014; 7:233). Animals were individually observed, and changes were recorded and scored according to their intensity. Observations included changes in appearance, activity, response to stimuli, eyes opening, breathing quality and evolution of body weight. For each of these clinical signs, the severity was measured on a scale of 0 to 3. The evolution of the severity was followed and the mean score at each time point was calculated and plotted on graphs. Arbitrary, when mice die or are euthanized, they are scored at 4.

### Results

NTZ treatments (with or without the 3-day pretreatment) greatly improved survival after CLP-induced sepsis (FIG. 5B). While mortality already reached 60% at 55 hours post-surgery in the control group, mortality in NTZ-treated mice with or without pretreatment only reached 25% and 17%, respectively. At the end of the study, 45.8% of mice with NTZ pretreatment, and 58.3% of mice with NTZ treatment survived sepsis compared to only 12.5% of untreated mice (FIG. 6).

The evolution of the severity of sepsis was assessed by welfare scoring. In all observed criteria, NTZ-treated mice (with or without pretreatment) had scores below the control mice, suggesting a lower global severity of sepsis and the improvement of animal welfare with NTZ (FIG. 7).

### Example 5: NTZ is a Potent Treatment of Sepsis Efficient in Curative Settings

Polymicrobial sepsis was induced by CLP surgery in C57BL6J mice, and NTZ was prepared and administrated per os, as described previously. In order to investigate the rapid effects of NTZ to counteract sepsis, NTZ was administrated after surgery i.e. when leakage of bacteria from gut microbiota in the peritoneum occurs. As shown in FIG. 8A, C57BI/6J male mice (8 week-old, Janvier, France) were divided into 3 groups of 20 mice each:
Group 1 received a first administration of vehicle 1 hour before surgery, then a second administration after surgery and twice daily for 6 days after CLP surgery.
Group 2 received a first administration of nitazoxanide (NTZ) 1 hour before surgery, then a second administration of NTZ 3.5 hours after surgery (2 times 50 mg/kg) and twice daily for 6 days after CLP surgery.
Group 3 received a first administration of NTZ (100 mg/kg) only 3.5 hours after surgery and then 50 mg/kg twice daily for 6 days after CLP surgery.

During the 6 days following the surgery, treatments with NTZ were made twice daily at 9 am and 5 pm at the dose of 100 mg/kg/day (p.o. BID) per mouse. Control mice received vehicle the same way to avoid any deviations between control and treated mice.

### Results

NTZ treatments starting on the day of CLP surgery, either before and after surgery (BID T-1 h/T+3.5 h), or only after surgery (QD T+3.5 h), had significant beneficial effects on survival (FIG. 8B). While mortality already reached 55% at 55 hours post-surgery in the control group, mortality in mice treated with NTZ BID T-1 h/T+3.5 h and QD T+3.5 h only reached 5% and 15%, respectively. At the end of the study (day 7), 80% and 70% of mice treated with NTZ BID T-1 h/T+3.5 h and QD T+3.5 h, respectively, survived sepsis compared to only 20% of untreated mice (FIG. 8C). The evolution of the severity of sepsis was also assessed by welfare scoring, as previously described. Mice that received QD T+3.5 h NTZ treatment had improved scores for all observed criteria, suggesting a lower global severity of sepsis and a great improvement of well-being after sepsis induction (FIG. 9).

### CONCLUSION

Taken together, these results show that NTZ is a very rapid and potent compound that protects from cell death and improves welfare and survival in sepsis.

1. A method of treating sepsis in a subject in need thereof, the method comprising administering to the subject a compound selected from the group consisting of nitazoxanide (NTZ), tizoxanide (TZ), TZ glucuronide (TZG), and combinations thereof.

2. The method according to claim 1, wherein said sepsis is caused by a bacterial infection.

3. The method according to claim 1, wherein the compound is used to protect vital organs by inhibiting cytokine-induced apoptosis that occurs during the transition from sepsis to septic shock.

4. The method according to claim 1, wherein the compound is used to protect against cytokine-induced cell death by inhibiting caspase activity.

5. The method according to claim 1, wherein said subject suffers from or is at risk of sepsis with multiple organ failure.

6. The method according to claim 1, wherein said subject suffers from or is at risk of septic shock.

7. The method according to claim 1, for use to slow or stop the progression of sepsis.

8. The method according to claim 1, wherein said compound is for use as a single active agent in said method.

9. The method according to claim 1, wherein said compound for use in combination with an antimicrobial agent in said method.

10. The method according to claim 9, wherein said antimicrobial agent is an antibiotic.

11. The method according to claim 9, wherein said antimicrobial agent is a carbapenem antibiotic.

12. The method according to claim 9, wherein said antimicrobial agent is ertapenem.

13. The method according to claim 1, wherein said compound is NTZ.
